(19) United States
(12) Patent Application Publication
(10) Pub. No.: US 2007/0020716 A1
(43) Pub. Date: Jan. 25, 2007

(54) Coronary Endothelial Dysfunction

(76) Inventors: Amir Lerman, Rochester, MN (US); Joseph P. McConnell, Rochester, MN (US)

Correspondence Address: FISH & RICHARDSON P.C., PO BOX 1022, MINNEAPOLIS, MN, 55440-1022, US

(21) Appl. No.: 11/457,736

(22) Filed: Jul. 14, 2006

Related U.S. Application Data
(60) Provisional application No. 60/699,236, filed on Jul. 14, 2005.

(51) Int. Cl. C12Q 1/34

(52) U.S. Cl. 435/18

(57) ABSTRACT

This document provides methods and materials related to assessing coronary endothelial dysfunction (CED) conditions as well as methods and materials related to treating coronary conditions. For example, methods and materials for determining whether or not a mammal has CED by determining whether or not a sample from the mammal contains an elevated level of a lipoprotein associated phospholipase A2 (Lp-PLA₂) polypeptide are provided.

### CROSS-REFERENCE TO RELATED APPLICATIONS

This application claims the benefit of U.S. Provisional Application Ser. No. 60/699,236, filed Jul. 14, 2005.

### STATEMENT AS TO FEDERALLY SPONSORED RESEARCH

This invention was made with government support under grant HL063911 awarded by the National Institutes of Health. The government has certain rights in the invention.

### BACKGROUND

1. Technical Field

This document relates to methods and materials related to assessing coronary endothelial dysfunction (CED) conditions as well as methods and materials related to treating coronary conditions.

2. Background Information

Coronary artery disease (CAD) is a common type of heart disease and a leading cause of morbidity and mortality. CAD occurs when the arteries that supply blood to the heart muscle (coronary arteries) become hardened and narrowed due to a buildup of plaque on the inner walls or lining of the arteries (a process called atherosclerosis). Blood flow to the heart can be reduced as plaque narrows the coronary arteries. This can decrease oxygen supply to the heart muscle and can cause angina and heart attack. Over time, CAD can weaken heart muscle and contribute to heart failure and arrhythmias.

### SUMMARY

This document provides methods and materials related to assessing coronary endothelial dysfunction (CED) conditions as well as methods and materials related to treating coronary conditions. For example, the methods and materials provided herein can be used to determine whether or not a sample from a mammal (e.g., a human) contains an elevated level of a lipoprotein associated phospholipase A2 (Lp-PLA₂) polypeptide. The presence of such an elevated level can indicate that the mammal has CED. Determining whether or not for example, humans have CED can help clinicians identify humans in need of medical treatment to help prevent or reduce the risk of experiencing coronary conditions or CAD. Once identified as having CED, a human can be treated with agents designed to prevent or reduce the risk of developing a CAD event. Treating humans having CED and no history of CAD can allow clinicians to reduce a person's risk of suffering from a potentially life threatening CAD event later in life.

In general, this document features a method for assessing coronary endothelial dysfunction in a human. The method includes determining whether or not a sample from the human has an elevated level of a lipoprotein associated phospholipase A2 polypeptide, wherein the human was not previously diagnosed with coronary artery disease, and wherein the presence of the elevated level indicates that the human has coronary endothelial dysfunction. The sample can be a blood sample. The sample can be a plasma sample. The elevated level can be a level greater than 180 ng of the lipoprotein associated phospholipase A2 polypeptide per mL of sample. The elevated level can be a level greater than 240 ng of the lipoprotein associated phospholipase A2 polypeptide per mL of sample. The method can include confirming whether or not the human has coronary endothelial dysfunction by determining whether or not intracoronary administration of acetylcholine increases coronary blood flow in the human to a level less than about 50 percent, wherein the presence of the increase to a level less than about 50 percent confirms that the human has coronary endothelial dysfunction. The method can include confirming whether or not the human has coronary endothelial dysfunction by determining whether or not intracoronary administration of acetylcholine increases coronary blood flow in the human to a level less than about 40 percent, wherein the presence of the increase to a level less than about 40 percent confirms that the human has coronary endothelial dysfunction. The method can include confirming whether or not the human has coronary endothelial dysfunction by determining whether or not intracoronary administration of acetylcholine decreases the coronary artery diameter in the human by greater than about 15 percent, wherein the presence of the decrease in coronary artery diameter by greater than about 15 percent confirms that the human has coronary endothelial dysfunction. The method can include confirming whether or not the human has coronary endothelial dysfunction by determining whether or not intracoronary administration of acetylcholine decreases the coronary artery diameter in the human by greater than about 20 percent, wherein the presence of the decrease in coronary artery diameter by greater than about 20 percent confirms that the human has coronary endothelial dysfunction.

In another aspect, this document features a method for reducing the risk of a coronary condition in a human. The method includes (a) determining whether or not a sample from the human has an elevated level of a lipoprotein associated phospholipase A2 polypeptide, wherein the human was not previously diagnosed with coronary artery disease, and wherein the presence of the elevated level indicates that the human has coronary endothelial dysfunction, and (b) administering, to the human found to have coronary endothelial dysfunction, an agent selected from the group consisting of statins, blood-thinning agents, inhibitors of a lipoprotein associated phospholipase A2 polypeptide, and cholesterol reuptake inhibitors. The sample can be a blood sample. The sample can be a plasma sample. The elevated level can be a level greater than 180 ng of the lipoprotein associated phospholipase A2 polypeptide per mL of sample. The elevated level can be a level greater than 240 ng of the lipoprotein associated phospholipase A2 polypeptide per mL of sample. Step (a) can include confirming whether or not the human has coronary endothelial dysfunction by determining whether or not intracoronary administration of acetylcholine increases coronary blood flow in the human to a level less than about 50 percent, wherein the presence of the increase to a level less than about 50 percent confirms that the human has coronary endothelial dysfunction. Step (a) can include confirming whether or not the human has coronary endothelial dysfunction by determining whether or not intracoronary administration of acetylcholine increases coronary blood flow in the human to a level less than about 40 percent, wherein the presence of the increase to a level less than about 40 percent confirms that the human has coronary endothelial dysfunction. Step (a) can include confirming whether or not the human has coronary endothelial dysfunction by determining whether or not intracoronary administration of acetylcholine decreases the coronary artery diameter in the human by greater than about 15 percent, wherein the presence of the decrease in coronary artery diameter by greater than about 15 percent confirms that the human has coronary endothelial dysfunction. Step (a) can include confirming whether or not the human has coronary endothelial dysfunction by determining whether or not intracoronary administration of acetylcholine decreases the coronary artery diameter in the human by greater than about 20 percent, wherein the presence of the decrease in coronary artery diameter by greater than about 20 percent confirms that the human has coronary endothelial dysfunction.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a bar graph plotting the unadjusted and adjusted percent change in coronary blood flow following intracoronary acetylcholine treatment for humans having 110-181.4 ng Lp-PLA₂ per mL plasma (Tertile 1), 181.48-239.6 ng Lp-PLA₂ per mL plasma (Tertile 2), or 240-443 ng Lp-PLA₂ per mL plasma (Tertile 3). The adjusted values were adjusted for age, sex, body mass index, serum creatinine, total cholesterol, LDL cholesterol, HDL cholesterol, triglycerides, and use of lipid lowering medication. p<0.001 for unadjusted and p=0.008 for adjusted data.

FIG. 2 is a bar graph plotting the unadjusted and adjusted percent change in coronary artery diameter following intracoronary acetylcholine treatment for humans having 110-181.4 ng Lp-PLA₂ per mL plasma (Tertile 1), 181.48-239.6 ng Lp-PLA₂ per mL plasma (Tertile 2), or 240-443 ng Lp-PLA₂ per mL plasma (Tertile 3). The adjusted values were adjusted for age, sex, body mass index, serum creatinine, total cholesterol, LDL cholesterol, HDL cholesterol, triglycerides, and use of lipid lowering medication. p<0.001 for unadjusted and p=0.006 for adjusted data.

FIG. 3 is a bar graph plotting serum Lp-PLA₂ levels in patients with normal and abnormal coronary endothelial function (p=0.001).

FIG. 4 is a graph plotting the odds ratio for coronary endothelial dysfunction and the indicated serum agent.

### DETAILED DESCRIPTION

This document provides methods and materials related to assessing CED conditions as well as methods and materials related to treating coronary conditions. For example, the methods and materials provided herein can be used to determine whether or not a mammal with or without a history of a coronary event (e.g., myocardial infarction, acute coronary syndrome, or a previous need for revascularization) has CED or is likely to develop a coronary condition or CAD. The mammal can be a human, non-human primate, goat, horse, cow, pig, dog, or cat. In some cases, the mammal is a healthy human with no history of a heart problem. A mammal can be classified as having CED or as being likely to develop a coronary condition or CAD by assessing the level of an Lp-PLA₂ polypeptide in a sample from the mammal. For example, a mammal found to have an elevated level of an Lp-PLA₂ polypeptide can be classified as having CED or as being likely to develop a coronary condition or CAD.

The term “elevated” as used herein with reference to an Lp-PLA₂ polypeptide level refers to any level of an Lp-PLA₂ polypeptide that is greater than either the level of an Lp-PLA₂ polypeptide found in a control sample or the average level of an Lp-PLA₂ polypeptide found in samples from a population of normal healthy mammals (e.g., a population of humans known to be free of CED). For example, when assessing a particular mammal for CED, the level of an Lp-PLA₂ polypeptide found in a blood sample from that mammal can be compared to the level of Lp-PLA₂ polypeptide found in blood samples obtained from a population of mammals known to be free of CED. Any population size can be used to determine the average level of an Lp-PLA₂ polypeptide found in samples from a population of normal healthy mammals. For example, a population of 2, 3, 4, 5, 10, 15, 20, 25, 30, 40, 50, or more humans free of CED can be used to determine the average level of an Lp-PLA₂ polypeptide in samples from a population of normal healthy mammals. An elevated level of an Lp-PLA₂ polypeptide can be 1, 2, 3, 4, 5, 10, 20, 30, 50, or more percent higher than that level found in a control sample or the average level of an Lp-PLA₂ polypeptide found in samples from a population of normal healthy mammals. In some cases, an elevated level of an Lp-PLA₂ polypeptide can be 1.1, 1.2, 1.5, 2, 3, 4, 5, 10, 50, 100, or more fold higher than that level found in a control sample or the average level of an Lp-PLA₂ polypeptide found in samples from a population of normal healthy mammals.

In some cases, an elevated level of an Lp-PLA₂ polypeptide in a human can be any level greater than 150 ng (e.g., greater than 160, 170, 180, 190, 200, 210, 220, 230, 240, 250, 260, 270, 280, 290, 300, 350, 400, 450, or more ng) of Lp-PLA₂ polypeptide per mL of sample. In some cases, an elevated level of an Lp-PLA₂ polypeptide in a human can be any level between 150 ng and 500 ng (e.g., between 180 ng and 500 ng, between 190 ng and 500 ng, between 200 ng and 500 ng, or between 200 and 400 ng) of Lp-PLA₂ polypeptide per mL of sample.

In some cases, a reference chart can be used to determine whether or not a particular level of an Lp-PLA₂ polypeptide in a sample is elevated, normal, or reduced. For example, a reference chart can contain the normal range of Lp-PLA₂ polypeptide levels found in samples from mammals with or without CED. Using this reference chart, any level of an Lp-PLA₂ polypeptide measured in a sample can be classified as being an elevated level, a normal level, or a reduced level.

As described herein, the presence of an elevated level of an Lp-PLA₂ polypeptide in a mammal's sample can indicate that the mammal has CED or is likely to develop a coronary condition or CAD. In the case of humans, an Lp-PLA₂ polypeptide can be a human Lp-PLA₂ polypeptide such as a polypeptide having the amino acid sequence set forth in Genbank Accession No. AAB04170. In the case of non-human mammals, an Lp-PLA₂ polypeptide can be a corresponding Lp-PLA₂ polypeptide. For example, in the case of Mus musculus, an Lp-PLA₂ polypeptide can be a mouse Lp-PLA₂ polypeptide such as a polypeptide having the amino acid sequence set forth in Genbank Accession No. 2201424C.

The level of an Lp-PLA₂ polypeptide within any sample can be determined. For example, the level of an Lp-PLA₂ polypeptide within a blood, serum, or plasma sample can be determined. Any method can be used to obtain a sample from a mammal. For example, blood samples can be obtained via venous puncture techniques using a needle and syringe. Serum samples can be prepared from whole blood using standard methods such as centrifuging blood samples that have been allowed to clot. Plasma samples can be obtained by centrifuging blood samples that were treated with an anti-coagulant such as heparin.

Any method can be used to determine the level of an Lp-PLA₂ polypeptide in a sample. For example, the level of an Lp-PLA₂ polypeptide can be determined by measuring the level of mRNA that encodes an Lp-PLA₂ polypeptide, by measuring the level of the Lp-PLA₂ polypeptide itself, or by measuring the level of Lp-PLA₂ polypeptide activity. Any method can be used to measure the level of mRNA that encodes an Lp-PLA₂ polypeptide including, without limitation, northern blotting techniques, slot blotting techniques, quantitative reverse transcriptase polymerase chain reaction (RT-PCR) techniques, or chip hybridization techniques. In addition, any method can be used to measure the level of the Lp-PLA₂ polypeptide itself including, without limitation, immunoassays such as ELISA or immunoblotting techniques. Any method can be used to measure the level of Lp-PLA₂ polypeptide activity including, without limitation, binding assays and radiometric activity assays. Prior to determining the level of an Lp-PLA₂ polypeptide in a sample, the sample can be subjected to one or more standard preparatory procedures.

Once identified as having CED or as being likely to develop a coronary condition or CAD, the mammal can begin treatment or can undergo additional testing to confirm or evaluate CED, a coronary condition, or CAD. For example, a mammal classified as having CED as described herein can undergo diagnostic tests such as coronary angiography, coronary blood flow measurements, or intravascular ultrasound to confirm the existence or severity of CED or to assess the existence or severity of CAD. In some cases, a mammal classified as having CED as described herein can initiate one or more treatments designed to reduce the likelihood of experiencing a coronary condition or CAD. Such treatments can include, without limitation, administering agents such as statins (e.g., simvastatin or pravastatin), blood-thinning agents (e.g., warfarin, enoxaparin sodium injection, or aspirin), Lp-PLA₂ polypeptide inhibitors, and cholesterol reuptake or absorption inhibitors (e.g., ezetimibe). Inhibitors of a lipoprotein associated phospholipase A2 polypeptide include, without limitation: 1-(biphenylmethylamidoalkyl)-pyrimidones, such as the diethylaminoethyl derivative SB-435495; compounds in which the pyrimidone 5-substituent in SB-435495 has been modified, including the cyclopentyl fused derivative SB-480848; 1-(Arylpiperazinylamidoalkyl)-pyrimidones; SB-253514, a natural product derived inhibitor and analogs thereof; and the azetidinone inhibitor SB-222657.

This document also provides kits that can be used to assess CED. For example, reagents used to determine the level of an Lp-PLA₂ polypeptide in a sample can be combined as an article of manufacture such as a kit. In one embodiment, a kit can contain reagents for the immunodetection of an Lp-PLA₂ polypeptide. For example, a kit can contain an anti-Lp-PLA₂ polypeptide antibody. In some cases, such a kit can include suitable reagents for detecting the binding of the anti-Lp-PLA₂ polypeptide antibody to an Lp-PLA₂ polypeptide with or without additional antibodies such as antibodies having the ability to bind an anti-Lp-PLA₂ polypeptide antibody. In some cases, a kit can contain buffers, positive control samples (e.g., a solution containing a known amount of an Lp-PLA₂ polypeptide), or combinations thereof. The reagents within a kit can be housed together in various combinations or can be packaged in separate vials or containers. The kits provided herein also can include labels or packaging inserts setting out instructions for preparation and use. For example, a kit can contain a packaging insert describing that the level of an Lp-PLA₂ polypeptide can be used to assess the presence or absence of CED.

The term “antibody” as used herein includes, without limitation, polyclonal, monoclonal, multi-specific, human, humanized, chimeric, and single-chain antibodies as well as antibody fragments having binding activity such as Fab fragments, F(ab′) fragments, F(ab′)2 fragments, Fd fragments, fragments produced by a Fab expression library, fragments comprising a VL or VH domain, and epitope binding fragments of any of the above. An antibody can be of any type (e.g., IgG, IgE, IgM, IgD, IgA, and IgY), class (e.g., IgG1, IgG2, IgG3, IgG4, IgA1, and IgA2), or subclass. In addition, antibodies can be from any animal including birds and mammals. For example, antibodies can be human, murine, rabbit, goat, guinea pig, camel, horse, or chicken.

An antibody can be naturally occurring, recombinant, or synthetic. Antibodies can be generated by any suitable method known in the art. For example, monoclonal antibodies can be prepared using a wide variety of techniques known in the art, including the use of hybridoma, recombinant, and phage display technologies, or a combination thereof An antibody such as an antibody fragment can be produced by any means. For example, an antibody fragment can be enzymatically or chemically produced by fragmentation of an intact antibody. An antibody fragment can also be produced synthetically or recombinantly from a gene encoding the partial antibody sequence.

Once an antibody has been produced by an animal, chemically synthesized, or recombinantly expressed, it can be purified by any method known in the art. For example, an antibody can be purified by chromatography, centrifugation, or differential solubility. In some cases, an antibody can be fused to a sequence, such as a hexa-histidine peptide, for convenient purification of the fusion protein.

Antibodies provided herein can be used, without limitation, to purify or detect a lipoprotein associated phospholipase A2 polypeptide. For example, the antibodies can be used in immunoassays for qualitatively and quantitatively measuring levels of lipoprotein associated phospholipase A2 polypeptides in biological samples (e.g., blood samples). The antibodies can be recombinantly fused or conjugated to molecules useful as labels in detection assays, such as enzymes, streptavidin, avidin, fluorescent molecules, luminescent molecules, bioluminescent molecules, and radioactive molecules. A detectable substance can be coupled or conjugated either directly to the antibody or fragment thereof, or indirectly, e.g., through a linker. In some cases, an antibody can be fused to another molecule (e.g., a polypeptide) to aid in the uptake of the antibody into cells. See, e.g., Mie et al., Biochem. Biophys. Res. Comm., 310(3): 730-734 (2003)).

As described herein, methods for assessing a mammal for coronary endothelial dysfunction (CED) based on the level of an Lp-PLA₂ polypeptide in the mammal are provided. Mammals that can be assessed for CED include mammals that are adults. For example, a human between the ages of about 18 and 85 (e.g., 25, 30, 35, 40, 45, 50, 55, 60, 65, 70, 75, 80, 85, or 90 years of age) can be assessed for CED. In addition, mammals that can be assessed for CED include mammals that were or were not previously diagnosed with coronary artery disease, as well as mammals that have CED, are suspected of having CED, or are susceptible to developing CED, such as mammals having a family history of a coronary condition.

A mammal can be assessed for CED by obtaining one or more samples, e.g., blood samples, from the mammal and determining the level or activity of an Lp-PLA₂ polypeptide in the sample(s). A sample can be obtained from a mammal that has not fasted, and/or a sample can be obtained from the mammal after the mammal has fasted, e.g., for about 5 to 24 hours. A sample can also be obtained from a mammal before, after, or at the time of a procedure, such as a coronary angiography.

A sample from a mammal can be processed and/or stored prior to analysis of the Lp-PLA₂ polypeptide level in the sample. For example, a sample of blood can be heparanized, centrifuged, and aliquoted into multiple containers prior to analysis. A sample can also be stored, e.g. at a temperature of −70° C. or colder, for analysis at a later time, such as weeks, months, or years after collection of the sample. In addition, a portion of a sample can be analyzed, and another portion of the same sample can be stored and analyzed again at a later point in time. Samples also can be obtained from a mammal over time, such as every three months, every six months, or once a year. Each sample can be divided into aliquots, an aliquot of each sample can be analyzed for an Lp-PLA₂ polypeptide level, and the remaining aliquot(s) can be stored.

A freshly obtained sample, or a sample that has been stored, can be analyzed for the level of an Lp-PLA₂ polypeptide as described herein. Measurement of the level of an Lp-PLA₂ polypeptide in a sample can be repeated multiple times, such as two, three, four or more times, and the replicate values can be averaged, or the mean value can be determined. A value from a single measurement, an average value, or a mean value can be used to assess CED. In addition, the Lp-PLA₂ polypeptide level in each of multiple samples from a mammal can be determined, and the values can be used independently for assessing CED, or the average or mean value can be used for assessing CED.

The level of an Lp-PLA₂ polypeptide in a sample from a mammal can be used to assess the mammal for CED by comparing the level to a reference level of an Lp-PLA₂ polypeptide or range of Lp-PLA₂ polypeptide levels, such as a level or range in a reference chart. A reference chart can contain Lp-PLA₂ polypeptide levels corresponding to those found in healthy mammals, e.g., mammals that do not have CED, and a level of an Lp-PLA₂ polypeptide in a sample from a mammal that is elevated relative to such a reference level can indicate that the mammal has CED. A reference chart also can contain Lp-PLA₂ polypeptide levels corresponding to those found in mammals that have CED, and a level of an Lp-PLA₂ polypeptide in a sample from a mammal that is lower than such a reference level can indicate that the mammal does not have CED. Levels of an Lp-PLA₂ polypeptide in samples collected from a mammal at different time points can be compared to determine if the level is increasing, decreasing, or remaining constant. An increase in the level of an Lp-PLA₂ polypeptide over time can indicate that the mammal has developed CED or is susceptible to develop CED. A decrease in the level of an Lp-PLA₂ polypeptide over time can indicate that a mammal having CED, or a susceptibility to develop CED, is improving.

Reference levels of an Lp-PLA₂ polypeptide, or ranges of levels of an Lp-PLA₂ polypeptide, corresponding to levels of an Lp-PLA₂ polypeptide in mammals that do and that do not have CED can be determined by measuring the levels of an Lp-PLA₂ polypeptide in samples, e.g., blood samples, from a random sampling of mammals. For example, the levels of an Lp-PLA₂ polypeptide in blood samples from a random sampling of 10 or more (e.g., 15, 25, 50, 75, 100, 150, 175, 200, 250, 300, 350, 400, 450, 500, 600, 700, 800, 900, 1000, 1500, 2000, 2500, 3000, 3500, 4000, 4500, 5000 or more) humans can be determined as described herein. A random sampling can include males and females having an age of about 48±15. In addition, a random sampling can include individuals having hypertension, diabetes, hyperlipidemia, a history of smoking, and/or an elevated blood pressure, as well as individuals who are overweight and/or have an elevated level of cholesterol or triglycerides. Individuals having significant obstructive coronary artery disease can be excluded from a random sampling. Individuals included in the random sampling can be evaluated to determine whether or not they have CED, e.g., by determining whether or not intracoronary administration of acetylcholine increases coronary blood flow to a level less than about 50 percent, as described herein.

A random sampling of mammals can be rank-ordered based on the level of an Lp-PLA₂ polypeptide in a sample from each mammal. The mammals can be divided into a selected number of groups, e.g., two, three, or four groups. For example, the mammals can be divided into three groups, or tertiles. For example, about one third of the mammals having the lowest levels of an Lp-PLA₂ polypeptide in their samples can comprise a first tertile, about one third of the mammals having intermediate levels of an Lp-PLA₂ polypeptide in their samples can comprise a second tertile, and about one third of the mammals having the highest levels of an Lp-PLA₂ polypeptide in their samples can comprise a third tertile. As described in Example 2, a first tertile can contain humans having an Lp-PLA₂ polypeptide level of 110-181.4 ng/mL of plasma, a second tertile can contain humans having an Lp-PLA₂ polypeptide level of 181.48-239.6 ng/mL of plasma, and a third tertile can contain humans having an Lp-PLA₂ polypeptide level of 240-443 ng/mL of plasma. In some cases, a first tertile can contain humans having an Lp-PLA₂ polypeptide level of about 100-170 ng/mL of plasma, a second tertile can contain humans having an Lp-PLA₂ polypeptide level of about 171-230 ng/mL of plasma, and a third tertile can contain humans having an Lp-PLA₂ polypeptide level of 230-450 ng/mL of plasma.

In some cases, mammals can be divided into three groups, and a grouping of mammals with the highest levels of an Lp-PLA₂ polypeptide can be characterized as having CED, a grouping with intermediate levels can be characterized as having a susceptibility to develop CED, and a grouping of mammals having the lowest levels of an Lp-PLA₂ polypeptide can be characterized as not having CED. In some cases, mammals in a random sampling can be evaluated for CED as described herein, and the association between the level of an Lp-PLA₂ polypeptide for a grouping of mammals and CED can be determined. A lower limit of a grouping of mammals characterized as having CED and/or an upper limit of a grouping of mammals characterized as not having CED, can be used to determine cutoff values or reference levels. For example, the upper and lower limits of a second tertile can be reference levels. In some cases, the upper limit of a second tertile can be used as a cutoff between mammals that have CED and mammals that are susceptible to develop CED or that do not have CED.

The association between a grouping (e.g., tertile) of Lp-PLA₂ polypeptide levels and endothelial function can be assessed using mathematical and/or statistical methods, e.g., linear regression models and linear contrast. The association can be assessed independent of or by including covariates, such as age, sex, body mass index, creatinine level, total cholesterol level, LDL cholesterol level, HDL cholesterol level, triglyceride level, and use of lipid lowering medication. Humans in tertile two and/or three can exhibit a significantly lower percentage change in coronary blood flow than humans in tertile one. In addition, humans in tertile two and/or three can exhibit a greater reduction in coronary artery diameter than those in tertile one. In some cases, the average percentage change in coronary blood flow and/or coronary artery diameter observed for humans in tertile three meet or exceed the criteria for defining CED, while the average percentage change in coronary blood flow and/or coronary artery diameter observed for humans in tertiles one and two do not meet the criteria for defining CED. In such cases, the lower limit of the range of Lp-PLA₂ polypeptide levels corresponding to tertile three can be a reference level of an Lp-PLA₂ polypeptide. A level of an Lp-PLA₂ polypeptide in a sample from a human that is lower than such a reference level can indicate that the human does not have CED, while a level that is equal to or greater than such a reference level can indicate that the human has CED. In addition, levels and/or ranges corresponding to tertiles one and two can serve as reference levels or ranges. For example, a level of an Lp-PLA₂ polypeptide in a sample from a human that falls within the range of Lp-PLA₂ polypeptide levels of tertile two can indicate that the human is susceptible to developing CED, and a level that is lower than a level corresponding to the lower limit of tertile two can indicate that the human does not have CED.

A reference level of an Lp-PLA₂ polypeptide or range of Lp-PLA₂ polypeptide levels corresponding to levels of an Lp-PLA₂ polypeptide found in mammals that do not have CED can be determined by measuring the levels of an Lp-PLA₂ polypeptide in samples from a random sampling of healthy mammals and using mathematical and/or statistical methods to analyze the data, e.g., to calculate a mean or median value, or a mean±standard deviation, or median and interquartile ranges, which can be reference levels or ranges. Samples from about 10 or more (e.g., 15, 25, 50, 75, 100, 150, 175, 200, 250, 300, 350, 400, 450, 500, 600, 700, 800, 900, 1000, 1500, 2000, 2500, 3000, 3500, 4000, 4500, 5000 or more) mammals can be analyzed to determine a reference level or range. A random sampling of healthy humans can include individuals between the ages of about 18 and 85. The health status of mammals included in the random sampling can be established, e.g., by performing a physical examination. In addition, tests can be performed that are indicative of a mammal's health, e.g., tests to determine cholesterol and/or blood sugar levels. In particular, it can be confirmed that mammals do not have coronary endothelial dysfunction, e.g., using methods described herein. One or more follow-up examinations can be performed, e.g., one, two, three, four, five, and/or ten years from the initial examination. The level of the Lp-PLA₂ polypeptide in a sample from any mammal found to have CED during a follow-up examination can be excluded or included from a determination of a reference level or range of Lp-PLA₂ polypeptide levels corresponding to levels of an Lp-PLA₂ polypeptide found in mammals who do not have CED.

A reference level of an Lp-PLA₂ polypeptide or range of Lp-PLA₂ polypeptide levels corresponding to levels of an Lp-PLA₂ polypeptide found in mammals who have CED can be determined by measuring the levels of an Lp-PLA₂ polypeptide in samples from a random sampling of mammals having CED (e.g., as determined using methods provided herein), and using mathematical and/or statistical methods to calculate a reference level(s) or range(s).

A reference level of an Lp-PLA₂ polypeptide or range of Lp-PLA₂ polypeptide levels corresponding to levels of an Lp-PLA₂ polypeptide found in mammals who are susceptible to developing CED can be determined by measuring the levels of an Lp-PLA₂ polypeptide in a random sampling of mammals who do not have CED and performing follow-up evaluations (e.g., every year, every two years, every five years, or every ten years) of the mammals for CED over time (e.g., over three years, five years, seven years, ten years, fifteen years, twenty years, or a lifetime). Once it has been determined which mammals in the random sampling developed CED and which mammals did not develop CED, then the levels of an Lp-PLA₂ polypeptide in the samples from the mammals who developed CED can be compared to the levels in the samples from the mammals who did not develop CED, and a reference level for susceptibility to developing CED can be determined. For example, samples from mammals that developed CED can have higher Lp-PLA₂ polypeptide levels than samples from mammals who did not develop CED, and the median or mean level of an Lp-PLA₂ polypeptide in samples from mammals who developed CED can be a reference level. Information about the susceptibility of a mammal to develop CED can be used to initiate treatment earlier than would otherwise be the case, and to guide treatment selection.

A mammal identified as having CED, or being susceptible to developing CED, can be treated to reduce the risk of a coronary condition or cardiovascular event, such as right ventricular hypertrophy or atherosclerosis. For example, a mammal identified as having CED can be treated using one or more of a statin, blood-thinning agent, an Lp-PLA₂ inhibitor, or a cholesterol absorption inhibitor. More than one agent can be used concomitantly or in succession. An agent can be administered in an amount, at a frequency, and for a duration effective to achieve a desired outcome (e.g., to reduce a symptom of CED) without producing significant toxicity to the mammal. In some cases, an agent can be administered to a mammal to improve a symptom (e.g., a decrease in the coronary artery diameter in response to intracoronary administration of acetylcholine) by 5, 10, 25, 50, 75, 99, or more percent. In some cases, the plasma level of an Lp-PLA₂ polypeptide can be used to determine whether or not an agent or combination of agents is effective. For example, the plasma level of an Lp-PLA₂ polypeptide can be determined prior to treatment and at various time points during treatment. A decrease in the plasma level of an Lp-PLA₂ polypeptide can indicate that the treatment is effective, while an increase in the plasma level of an Lp-PLA₂ polypeptide can indicate that the treatment is not effective.

Effective amounts of therapeutic agents can depend on various factors, such as the activities of the particular agents used, the frequency of administration, the duration of treatment, the severity of the condition being treated, and the condition and prior medical history of the mammal being treated. In some cases, a commonly prescribed amount of a statin, blood-thinning agent, Lp-PLA₂ inhibitor, or cholesterol absorption inhibitor can be used. In some cases, a commonly prescribed amount can be used to estimate an effective dose. A dose that is lower than an effective dose can initially be administered to a mammal, and the dose can then be gradually increased over time until the desired effect is achieved.

The frequency and duration of administration can be any frequency or duration that improves a symptom of CED without being toxic. For example, an agent can be administered once or twice a day, once every other day, once or twice a week, or as needed. The frequency of administration can remain constant or can be variable during the duration of treatment. An effective duration of treatment can vary from several weeks to several months or years. For example, an effective duration of treatment can be six months, five years, or a lifetime. In addition, a course of treatment can include rest periods. Multiple factors can influence the actual effective frequency and duration of treatment. For example, the activities of the particular therapeutic agents used, the severity of the condition being treated, the doses administered, and the condition and prior medical history of the mammal being treated can affect the effective frequency and duration of treatment.

The information concerning the level of an Lp-PLA₂ polypeptide in a mammal, or information that a mammal does or does not have CED, or is or is not susceptible to developing CED, can be recorded in a tangible or non-tangible form, or can be communicated or reported to another person. Any method can be used to communicate information to another person. For example, information can be given directly or indirectly to a person. In addition, any type of communication can be used to communicate the information. For example, mail, e-mail, telephone, and face-to-face interactions can be used. The information also can be communicated to a person by making that information electronically available to the person. For example, the information can be communicated to a person by placing the information on a computer database such that the person can access the information. In addition, the information can be communicated to a hospital, clinic, or research facility at which the person is located.

The invention will be further described in the following examples, which do not limit the scope of the invention described in the claims.

### EXAMPLES

### Example 1

### Experimental and Statistical Methods

Patients with a ≧30% epicardial coronary artery stenosis, coronary artery bridging in any segment of the left anterior descending coronary artery, or an ejection fraction <40% were excluded. Patient characteristics and past medical history were obtained from review of medical records. Blood samples were drawn in the fasting state 48 hours prior to the procedure and at the time of coronary angiography. Systemic blood pressure was invasively measured at the time of cardiac catheterization. Patients were instructed to not take any vasoactive medications within 48 hours of coronary angiography and endothelial function testing.

After diagnostic angiography and exclusion of significant obstructive coronary artery disease, endothelium-dependent coronary vasoactivity was assessed as described elsewhere (Suwaidi et al., Circulation, 101:948-954 (2000) and Hasdai et al., Circulation, 96:3390-3395 (1997)). In brief, a Doppler guidewire (Flowire, Volcano Inc.) within a coronary-infusion catheter (Ultrafuse, SciMed Life System) was positioned into the midportion of the left anterior descending coronary artery. Intra-coronary acetylcholine at increasing concentrations of 10⁻⁶, 10⁻⁵, and 10⁻⁴ M was infused to obtain effective coronary concentrations of 10⁻⁸, 10⁻⁷, and 10⁻⁶ M, respectively. Each dose of acetylcholine was infused for three minutes through the infusion catheter into the left anterior descending coronary artery to assess endothelium dependent vasoreactivity. Hemodynamic data, Doppler measurements, and a coronary angiogram were obtained after each infusion. Coronary artery diameter was measured by an independent investigator in the segment 5 mm distal to the tip of the Doppler wire using a computer-based image analysis system. Average peak velocity (APV) was derived from the Doppler flow velocity spectra, and coronary blood flow (CBF) was determined as π(coronary artery diameter/2)²×(APV/2). According to previous studies, microvascular endothelial dysfunction was defined as an increase in CBF ≦50%, and epicardial endothelial dysfunction was defined as a change in epicardial coronary artery diameter ≦−20% in response to the maximum dose of acetylcholine (Suwaidi et al., Circulation, 101:948-954 (2000) and Hasdai et al., Circulation, 96:3390-3395 (1997)). Endothelium-independent microvascular function was determined by the coronary flow reserve (CFR), which is the ratio of the APV at maximal hyperemia to the APV at baseline. Intracoronary adenosine (18-48 μg) was used to induce hyperemia, and a CFR ≦2.5 was considered abnormal (Kern, Catheterization and Cardiovascular Interventions, 54:378-400 (2001)). Lp-PLA₂ levels were measured in plasma aliquots, which were obtained at the time of coronary angiography and stored at −70° C., using an enzyme-linked immunoassay (PLAC™ test, diaDexus, Inc; Caslake et al., Atherosclerosis, 150:413-419 (2000)). Samples were incubated in microtitre plate wells with immobilized monoclonal antibody (2C10) against Lp-PLA₂. A second monoclonal antibody (4B4) labeled with horseradish peroxidase was used to identify the enzyme, and recombinant Lp-PLA₂ was used as the standard reference. The range of detection was 50-1000 ng/mL, and the interassay coefficients of variation were 7.8% at 276 ng/mL, 6.1% at 257 ng/mL, and 13.5% at 105 ng/mL. The 2C10 monoclonal antibody against Lp-PLA₂ has been shown to have no cross-reactivity with other A₂ phospholipases (Caslake et al., Atherosclerosis, 150:413-419 (2000)). All assays were performed by a single investigator who was blinded to the clinical characteristics and results of endothelial function assessment.

Patients were divided into three groups defined by the tertiles of the Lp-PLA₂ distribution. Continuous variables with little to mild skew were presented as mean±standard deviation, and skewed measures were presented as median and inter-quartile ranges (IQR). Discrete variables were summarized as frequencies and within group percentages. A trend for group differences across the three groups was tested using linear contrasts in association with one-way ANOVA for continuous variables (with log-transformation of the skewed variables) and the Armitage test for trend for discrete variables.

The association between Lp-PLA₂ tertiles and endothelial function independent of other covariates was assessed using linear regression models. Linear contrast was used to test for trend in endothelial function across Lp-PLA₂ tertiles. Covariates were selected for model adjustment if they were significantly associated with Lp-PLA₂ at the 0.10 significance level. The correlation between continuous covariates and Lp-PLA₂ was measured with Spearman's rank correlation coefficient. The associations between dichotomous variables and logged values of Lp-PLA₂ were tested with Student's t-tests. Age was not significantly associated with Lp-PLA₂, but was included for clinical relevance. The covariates included in the models were age, sex, body mass index, creatinine (log-transformed), total cholesterol, LDL cholesterol, HDL cholesterol, triglycerides (log-transformed), and use of lipid lowering medication.

### Example 2

### Elevated Lp-PLA₂ Levels Correlate with the Presence of CED

One hundred seventy two patients with Lp-PLA₂ concentrations ranging from 110 to 443 ng/mL were studied. Patients were divided into three groups based on the following tertiles of Lp-PLA₂: Tertile 1 (110-181.4 ng/mL, N=57), Tertile 2 (181.48-239.6 ng/mL, N=58), and Tertile 3 (240-443 ng/mL, N=57). Compared to patients in the lowest tertile (Table 1), there was a significant trend for a greater percentage of men in tertiles 2 and 3 (47% and 54% versus 14%, p<0.001) and a greater serum creatinine (1.0 [IQR, 0.9-1.1] and 1.1 [0.9-1.2] versus 1.0 [0.8-1.0] mg/dL, p=0.013). Patients in tertiles 2 and 3 also exhibited a significant trend for a greater total cholesterol (181.3±43.4 and 193.3±37.1 versus 169.2±36.0 mg/dL, p=0.001), higher LDL (106.1±35.5 and 115.9±29.5 versus 88.6±27.7 mg/dL, p<0.001), lower HDL (48.7±16.2 and 46.5±15.5 versus 56.2±15.6 mg/dL, p=0.001), and higher triglycerides (121.5 [IQR, 87-160] and 125.0 [78-205] versus 98 [60-147] mg/dL, p=0.005). There was no sign difference between the three groups in terms of age, prevalence of hypertension and diabetes, smoking history, C-reactive protein, and use of cardiac medications (aspirin, beta blockade, angiotensin converting enzyme inhibitors, and lipid lowering agents). The coronary flow reserve was not significantly different across the three groups.

TABLE 1
Patient Characteristics

| | Tertile 1 | Tertile 2 | Tertile 3 | |
|---|---|---|---|---|
| | 110-181.4 ng/mL | 181.48-239.6 ng/mL | 240-443 ng/mL | |
| | (N = 57) | (N = 58) | (N = 57) | P-value* |
| Age | 50.1 ± 13.2 | 48.1 ± 12.0 | 48.3 ± 10.7 | 0.41 |
| Male, No. (%) | 8 (14) | 27 (47) | 31 (54) | <0.001 |
| Hypertension, No. (%) | 26 (46) | 21 (37) | 29 (51) | 0.57 |
| Diabetes, No. (%) | 3 (5) | 5 (9) | 7 (12) | 0.18 |
| Hyperlipidemia, No. (%) | 36 (64) | 33 (57) | 35 (61) | 0.76 |
| History of Smoking, No. (%) | 21 (37) | 21 (36) | 24 (42) | 0.56 |
| Body mass index (kg/m²) | 27.6 + 5.8 | 28.6 ± 5.6 | 29.4 ± 5.9 | 0.10 |
| Mean arterial pressure (mm Hg), | 95.0 | 97.5 | 94.0 | 0.94 |
| Median (IQR) | (89.0-103.0) | (87.0-107.0) | (85.0-109.0) | |
| Creatinine (mg/dl), | 1.0 | 1.0 | 1.1 | 0.013 |
| Median (IQR) | (0.8-1.0) | (0.9-1.1) | (0.9-1.2) | |
| Total cholesterol (mg/dL) | 169.2 ± 36.0 | 181.3 ± 43.4 | 193.3 ± 37.1 | 0.001 |
| LDL cholesterol (mg/dL) | 88.6 ± 27.7 | 106.1 ± 35.5 | 115.9 ± 29.5 | <0.001 |
| HDL cholesterol (mg/dL) | 56.2 ± 15.6 | 48.7 ± 16.2 | 46.5 ± 15.5 | 0.001 |
| Triglycerides (mg/dL), | 96.0 | 121.5 | 125.0 | 0.005 |
| Median (IQR) | (60.0-147.0) | (87.0-160.0) | (78.0-205.0) | |
| C-reactive protein (mg/dL), | 0.2 | 0.2 | 0.3 | 0.89 |
| Median (IQR) | (0.1-0.7) | (0.1-0.6) | (0.1-0.6) | |
| Coronary flow reserve, | 2.9 | 3.0 | 3.0 | 0.34 |
| Median (IQR) | (2.6-3.3) | (2.5-3.5) | (2.6-3.5) | |

IQR = inter-quartile range,
*test for trend

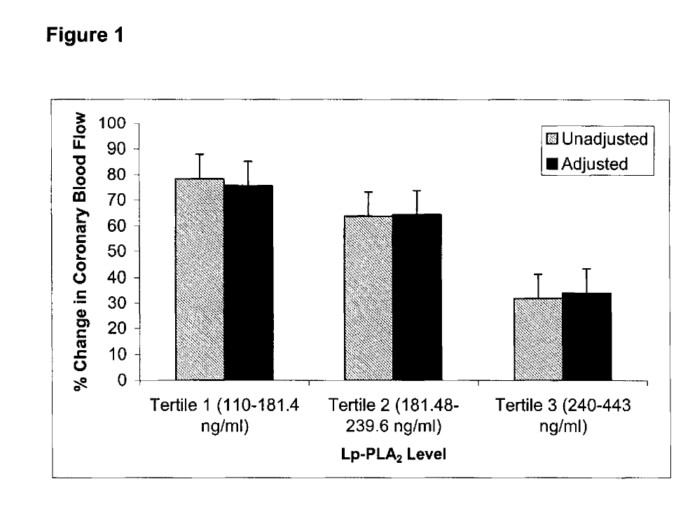

Microvascular endothelial function as determined by percentage change in coronary blood flow in response to the maximal dose of intracoronary acetylcholine was measured (FIG. 1). Patients in tertiles 2 and 3 exhibited a significantly lower percentage change in coronary blood flow than those in tertile 1 (63.8±73.2 and 32.0±71.7 versus 78.4±15.2%, p<0.001). This difference remained significant (p=0.008) after adjusting for age, sex, body mass index, serum creatinine, total cholesterol, LDL cholesterol, HDL cholesterol, triglycerides, and use of lipid lowering medications.

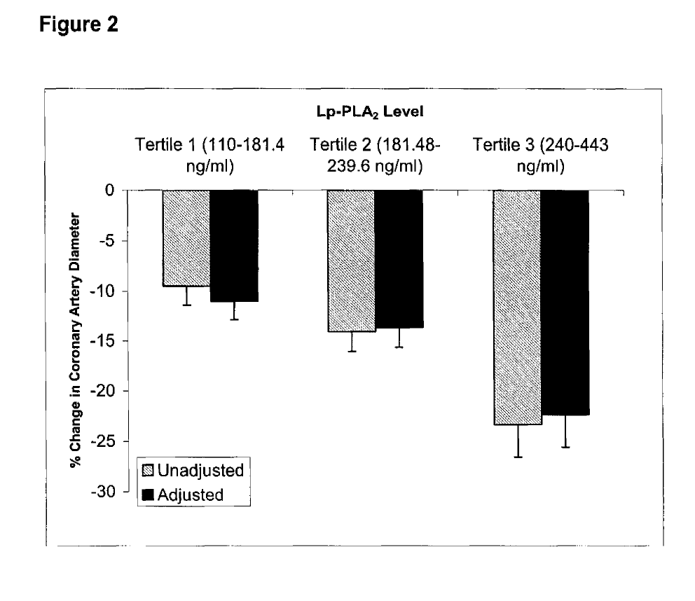

Epicardial endothelial function as determined by percentage change in coronary artery diameter in response to the maximal dose of acetylcholine was measured (FIG. 2). Patients in tertiles 2 and 3 exhibited a greater reduction in coronary artery diameter than those in the lowest tertile (−14.1±14.7 and −23.3±25.1 versus −9.5±15.2%, p<0.001). After adjusting for age, sex, body mass index, serum creatinine, total cholesterol, LDL cholesterol, HDL cholesterol, triglycerides, and use of lipid lowering medications the trend remained significant (p=0.006).

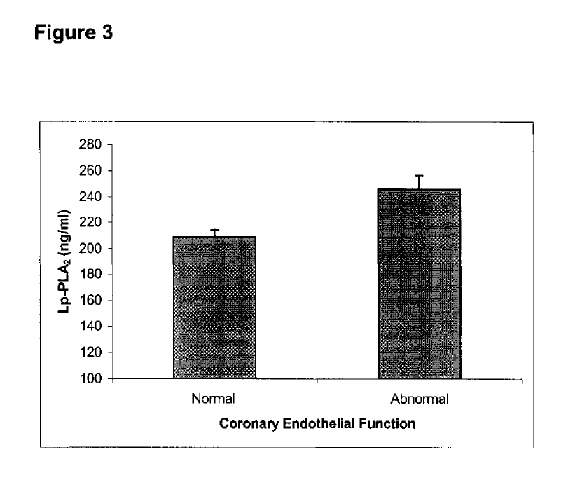

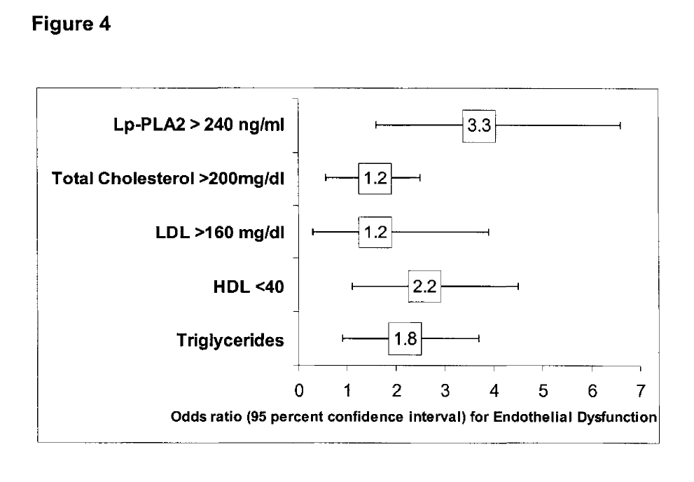

Patients with coronary endothelial dysfunction exhibited a significantly higher serum concentration of Lp-PLA₂ (209±56.7 versus 246.2±71.6, p=0.001; FIG. 3). The odds ratio of coronary endothelial dysfunction (FIG. 4) for patients in the highest tertile of Lp-PLA₂ was 3.3 (95 percent confidence interval 1.6-6.6).

### Example 3

### Measurement of Lp-PLA₇ Activity

Plasma aliquots prepared from blood samples (e.g., non-fasting blood samples) are collected and stored at −80° C., and Lp-PLA₂ activity is measured using a high throughput radiometric activity assay. Briefly, plasma samples are divided into aliquots, placed in 96-well microtiter plates, and mixed with a substrate solution consisting of 0.4 μmol/L [³H]-platelet activating factor (PAF) having a specific activity of about 20 Ci/mmol, and 99.6 μmol/L C16-PAF in assay buffer (100 mmol/L HEPES, 150 mmol/L NaCl, 5 mmol/L EDTA, pH 7.4). The reactions are allowed to proceed at room temperature for five minutes before sequestering the phospholipid substrates using an ice-cold, fatty acid-free BSA solution at a final concentration of 16.1 mg/mL. The BSA-lipid complexes are precipitated with ice-cold trichloroacetic acid at a final concentration of 7.8% and pelleted by centrifugation at about 6000 g for 15 minutes at 4° C. Aliquots of the supernatant containing the reaction products are transferred to another microplate, and radioactivity is counted in a Topcount liquid scintillation counter (e.g., PerkinElmer Life and Analytical Sciences, Wellesley, Mass.) on addition of scintillation cocktail (e.g., Microscint-20, PerkinElmer Life and Analytical Sciences). Lp-PLA₂ activity is expressed as nanomoles of PAF hydrolyzed per minute per one mL of plasma sample.

In another experiment, Lp-PLA₂ activity is measured using [³H]-PAF as a substrate. The assays are performed at 37° C. in 50 mmol/L HEPES and 150 mmol/L NaCl, pH 7.4. Plasma samples containing Lp-PLA₂ are mixed with [³H]-PAF (e.g., 20 nM) in a final volume of 200 μL and incubated for 10 minutes at 37° C. The reaction is stopped by vortexing with 600 μL of CHCl₃/MeOH (2:1), and the CHCl₃ and aqueous layers are separated by centrifugation (e.g., at about 12,000 g for about 15 minutes). An aliquot of the upper aqueous layer (250 μL) is removed to a fresh tube and vortexed with 250 μL of CHCl₃. The upper aqueous layer is again removed to a fresh tube and the amount of [31H] acetate is determined by scintillation counting. Reaction volumes are scaled up or down as appropriate to optimize the [3H] acetate signal level.

In another experiment, Lp-PLA₂ polypeptides are isolated from blood or plasma, e.g., on a polypeptide affinity column, by immunoprecipitation using an anti-Lp-PLA₂ antibody, or using HPLC, and the Lp-PLA₂ activity assay is performed as described above.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing coronary endothelial dysfunction in a human, said method comprising determining whether or not a sample from said human has an elevated level of a lipoprotein associated phospholipase A2 polypeptide, wherein said human was not previously diagnosed with coronary artery disease, and wherein the presence of said elevated level indicates that said human has coronary endothelial dysfunction.

2. The method of claim 1, wherein said sample is a blood sample.

3. The method of claim 1, wherein said sample is a plasma sample.

4. The method of claim 1, wherein said elevated level is a level greater than 180 ng of said lipoprotein associated phospholipase A2 polypeptide per mL of sample.

5. The method of claim 1, wherein said elevated level is a level greater than 240 ng of said lipoprotein associated phospholipase A2 polypeptide per mL of sample.

6. The method of claim 1, wherein said method further comprises confirming whether or not said human has coronary endothelial dysfunction by determining whether or not intracoronary administration of acetylcholine increases coronary blood flow in said human to a level less than about 50 percent, wherein the presence of said increase to a level less than about 50 percent confirms that said human has coronary endothelial dysfunction.

7. The method of claim 1, wherein said method further comprises confirming whether or not said human has coronary endothelial dysfunction by determining whether or not intracoronary administration of acetylcholine increases coronary blood flow in said human to a level less than about 40 percent, wherein the presence of said increase to a level less than about 40 percent confirms that said human has coronary endothelial dysfunction.

8. The method of claim 1, wherein said method further comprises confirming whether or not said human has coronary endothelial dysfunction by determining whether or not intracoronary administration of acetylcholine decreases the coronary artery diameter in said human by greater than about 15 percent, wherein the presence of said decrease in coronary artery diameter by greater than about 15 percent confirms that said human has coronary endothelial dysfunction.

9. The method of claim 1, wherein said method further comprises confirming whether or not said human has coronary endothelial dysfunction by determining whether or not intracoronary administration of acetylcholine decreases the coronary artery diameter in said human by greater than about 20 percent, wherein the presence of said decrease in coronary artery diameter by greater than about 20 percent confirms that said human has coronary endothelial dysfunction.

10. A method for reducing the risk of a coronary condition in a human, said method comprising:
(a) determining whether or not a sample from said human has an elevated level of a lipoprotein associated phospholipase A2 polypeptide, wherein said human was not previously diagnosed with coronary artery disease, and wherein the presence of said elevated level indicates that said human has coronary endothelial dysfunction, and
(b) administering, to said human found to have coronary endothelial dysfunction, an agent selected from the group consisting of statins, blood-thinning agents, inhibitors of a lipoprotein associated phospholipase A2 polypeptide, and cholesterol reuptake inhibitors.

11. The method of claim 1O, wherein said sample is a blood sample.

12. The method of claim 10, wherein said sample is a plasma sample.

13. The method of claim 10, wherein said elevated level is a level greater than 180 ng of said lipoprotein associated phospholipase A2 polypeptide per mL of sample.

14. The method of claim 10, wherein said elevated level is a level greater than 240 ng of said lipoprotein associated phospholipase A2 polypeptide per mL of sample.

15. The method of claim 10, wherein step (a) further comprises confirming whether or not said human has coronary endothelial dysfunction by determining whether or not intracoronary administration of acetylcholine increases coronary blood flow in said human to a level less than about 50 percent, wherein the presence of said increase to a level less than about 50 percent confirms that said human has coronary endothelial dysfunction.

16. The method of claim 10, wherein step (a) further comprises confirming whether or not said human has coronary endothelial dysfunction by determining whether or not intracoronary administration of acetylcholine increases coronary blood flow in said human to a level less than about 40 percent, wherein the presence of said increase to a level less than about 40 percent confirms that said human has coronary endothelial dysfunction.

17. The method of claim 10, wherein step (a) further comprises confirming whether or not said human has coronary endothelial dysfunction by determining whether or not intracoronary administration of acetylcholine decreases the coronary artery diameter in said human by greater than about 15 percent, wherein the presence of said decrease in coronary artery diameter by greater than about 15 percent confirms that said human has coronary endothelial dysfunction.

18. The method of claim 10, wherein step (a) further comprises confirming whether or not said human has coronary endothelial dysfunction by determining whether or not intracoronary administration of acetylcholine decreases the coronary artery diameter in said human by greater than about 20 percent, wherein the presence of said decrease in coronary artery diameter by greater than about 20 percent confirms that said human has coronary endothelial dysfunction.
